(19) United States
(12) Patent Application Publication
(10) Pub. No.: US 2022/0192544 A1
(43) Pub. Date: Jun. 23, 2022

(54) METHOD FOR DETERMINING A CURRENT GLUCOSE VALUE IN A TRANSPORTED FLUID

(71) Applicant: EYESENSE GMBH, Grossostheim (DE)

(72) Inventors: Theresa KRUSE, Ulm (DE); Knut GRAICHEN, Blaubeuren (DE); Achim MUELLER, Grossostheim (DE); Roland KRIVANEK, Grossostheim (DE)

(73) Assignee: EYESENSE GMBH, Grossostheim (DE)

(21) Appl. No.: 17/603,741

(22) PCT Filed: Apr. 9, 2020
(86) PCT No.: PCT/DE2020/200027

§ 371 Date: Oct. 14, 2021

(30) Foreign Application Priority Data
Apr. 15, 2019 (DE) 10 2019 205 430.7

(51) Int. Cl. A61B 5/145; A61B 5/00; A61B 5/1459

(52) U.S. Cl. CPC A61B 5/14532; A61B 5/7203; A61B 2562/0238; A61B 5/1459; A61B 5/1451; A61B 5/725

(57) ABSTRACT

The invention relates to a method in particular for continuously determining a current glucose value in a transported fluid, in particular blood, of an organism, having the steps of: a) ascertaining a series of measurements, comprising at least two measurement values separated by time intervals, for a tissue glucose value in the tissue surrounding the transported fluid using a sensor device, b) ascertaining the tissue glucose value using the ascertained series of measurements on the basis of a sensor model, wherein measurement values of the sensor device are assigned to tissue glucose values while taking into consideration measurement noise using a sensor model, c) providing a state transition model, the ascertained tissue glucose values being assigned at least one glucose value in the transported fluid using the state transition model while taking into consideration process noise, and d) ascertaining the current glucose value on the basis of the provided state transition model and the ascertained tissue glucose value. At least step d), in particular steps b)-d), is carried out using at least one moving horizon estimation method.

The invention relates to a process to, preferably continuously, determine a current glucose level in a transport fluid, particularly blood, of an organism.

The invention further relates to a device to, preferably continuously, determine a current glucose level in a transport fluid, particularly blood, of an organism.

The invention furthermore relates to an evaluating device to, preferably continuously, determine a current glucose level in a transport fluid, particularly blood, of an organism.

The invention further relates to a non-tangible, machine-readable medium for storing instructions that, when carried out on a computer, cause a process to be carried out to, preferably continuously, determine a current glucose level in a transport fluid, particularly blood, of an organism.

Although the present invention can be generally applied to any processes to determine a current glucose level in a transport fluid, the present invention is explained with regard to the blood glucose concentration in an organism.

To determine a blood glucose concentration BG in an organism, particularly in humans, systems for continuous glucose monitoring, also called CGM—Continuous Glucose Monitoring, have become known. In a CGM system, typically an interstitial tissue glucose concentration IG is automated, for example, measured every one to five minutes. In particular, diabetes patients benefit from CGM systems, because, in comparison to self-monitoring processes—also called Self Monitoring Processes—in which the patient himself determines the blood glucose level manually four to ten times a day, measurements can be carried out with significantly higher frequency. This allows for automated evaluations and warning signals to the patient, particularly even while the patient is sleeping, which helps to prevent critical health conditions in patients.

Known CGM systems are based, on the one hand, on electro-chemical processes. A CGM system of this kind is described in WO 2006/017358 A1, for example. Furthermore, optical CGM systems have become known, for example from DE 10 2015 101 847 B4, in which fluorescence dependent on glucose level is used and which is hereby included by way of reference. Both kinds of CGM systems measure an interstitial tissue glucose concentration.

Furthermore, it is known that the tissue glucose concentration or interstitial glucose (IG) concentration deviates from the blood glucose concentration, hereinafter abbreviated as BG. There is a large deviation particularly after strong influences on the blood glucose level, for example through the intake of food or nutrients or the supplying of insulin, as described in the non-patent literature Basu, Ananda et al. “Time lag of Glucose from intravascular to interstitial compartment in humans.” (Diabetes (2013): DB-131132). This deviation is caused by a diffusion process in the tissue surrounding the blood, so that the IG level is delayed in time and follows the BG level in a muffled manner, for example, as described in the non-patent literature Rebrin, Kerstin et al. “Subcutaneous Glucose predicts plasma Glucose independent of insulin: implications for continuous monitoring” (American Journal of Physiology-Endocrinology and Metabolism 277.3 (1999): E561-E571).

Because of the muffling and time-delay between the two glucose concentrations as described, on the one hand in the blood BG, and on the other hand in the surrounding tissue (IG), a calibration of the CGM system through a manual determination of the blood glucose concentration, for example by extracting a drop of blood from the finger and determining the glucose concentration in the drop of blood using an external measuring device, leads to significant inaccuracies.

In order to achieve an accurate calibration of the CGM system, however, the above-described difference between the tissue glucose concentration and the blood glucose concentration must be taken into account or at least assessed. To do this, various processes have become known. From the non-patent literature, Keenan, D. Barry et al. “Delays in minimally invasive continuous Glucose monitoring devices: a review of current technology.” (Journal of diabetes science and technology 3.5 (2009): 1207-1214), using a time-delayed glucose signal for calibration, has become known. Furthermore, from the non-patent literature Knobbe, Edward J. and Bruce Buckingham “The extended Kalman filter for continuous Glukose monitoring.” (Diabetes technology & therapeutics 7.1 (2005): 15-27), it has become known how to compensate for the muffling and the time delay of the diffusion process of glucose between the blood and the tissue using a Kalman filter.

An objective of the present invention, therefore, is to provide a device and also an evaluating device, which enables a more accurate determination of the glucose level, particularly in blood, with higher flexibility at the same time, particularly in relation to taking into account additional parameters and simpler implementation. A further objective of the present invention is to provide an alternative process, an alternative device and also an alternative evaluating device. A further objective of the present invention is to provide a process, a device and also an evaluating device with improved determination of the blood glucose concentration in an organism based on measuring the interstitial tissue glucose level.

In one embodiment, the present invention solves the above-mentioned objectives by a process to, preferably continuously, determine a current glucose level in a transport fluid, particularly blood, of an organism, comprising the steps
a) To determine, using a sensor device, a series of measurements, comprising at least two measurements separated in time for a tissue glucose level, in the tissue surround the transport fluid,
b) To determine the tissue glucose level using the series of measurements given, based on a sensor model, in which, by means of a sensor model, measurements of the sensor device are correlated to the tissue glucose levels while taking into account measurement noise,
c) To provide a state transition model, in which, by means of the state transition model, at least one glucose level in the transport fluid is correlated to the tissue glucose levels that have been determined while taking into account process noise, and
d) To determine the current glucose level based on the state transition model that has been provided and the tissue glucose level that has been determined, in which, at least step d), particularly steps b)-d), is carried out using at least one Moving Horizon Estimation Method.

In a further embodiment, the present invention solves the above-mentioned objectives by a device to, preferably continuously, determine a current glucose level in a transport fluid, particularly blood, of an organism, preferably suitable for carrying out a process according to one of claims 1-17, comprising

a sensor device, particularly for measuring fluorescence in a tissue surrounding the transport fluid, by means of a fiber optic probe, designed to determine a series of measurements, comprising at least two measurements separated in time for a tissue surrounding the transport fluid,
a provision device designed to provide a state transition model, in which, by means of the state transition model, at least one glucose level in the transport fluid is correlated to the determined tissue glucose levels while taking into account process noise, and to provide a sensor model, in which, by means of a sensor model, measurements of the sensor device are correlated to tissue glucose levels taking into account measurement noise,

An evaluating device designed to determine the tissue glucose level, using the series of measurements provided, based on the sensor model and to determine the current glucose level based on the state transition model that has been provided and the tissue glucose level that has been ascertained, using a Moving Horizon Estimation Method.

In one further embodiment, the present invention solves the above-mentioned objectives by an evaluating device to, preferably continuously, determine a current glucose level in a transport fluid, particularly blood, of an organism, preferably suitable for carrying out a process according to one of claims 1-17, comprising

at least one interface to connect a sensor device to provide a series of measurements, comprising at least two measurements separated in time for a tissue glucose level, in which the tissue surrounding the transport fluid,
at least one memory to store a state transition model, in which, at least one glucose level in the transport fluid is correlated to the tissue glucose levels by means of the state transition model while taking into account at least one process noise level, and to store a sensor model, in which, by means of the sensor model, measurements of the sensor device are correlated to tissue glucose levels while taking into account at least one measurement noise, and

A calculating device designed to determine the tissue glucose level, using the series of measurements provided, based on the sensor model and to determine the current glucose level based on the state transition model that has been stored and the tissue glucose level that has been determined, using at least one Moving Horizon Estimation Method.

In one further embodiment, the present invention solves the above-mentioned objectives through a non-tangible, machine-readable medium for the storage of instructions, which, when carried out on a computer, cause a process, particularly, to continuously determine a current glucose level in a transport fluid, particularly blood, of an organism, to be carried out preferably suitable for carrying out a process according to one of claims 1-17, comprising the steps
a) To determine, using a sensor device, a series of measurements, comprising at least two measurements separated in time for a tissue glucose level, in the tissue surround the transport fluid,
b) To determine the tissue glucose level using the series of measurements given, based on a sensor model, in which, by means of a sensor model, measurements of the sensor device are correlated to the tissue glucose levels while taking into account measurement noise,
c) To provide a state transition model, in which, by means of the state transition model, at least one glucose level in the transport fluid is correlated to the tissue glucose levels that have been determined while taking into account process noise, and
d) To determine the current glucose level based on the state transition model that has been provided and the tissue glucose level that has been determined,
   in which, at least step d), particularly steps b)-d), is carried out using at least one Moving Horizon Estimation Method.

In other words, a process to determine a blood glucose concentration in an organism is proposed. The latter presents the following process steps:

in a first process step, there is a series of measurements with at least two sensor measurements—spaced in time—of a tissue glucose level of the tissue of the organism by means of a sensor. In a further step, a sensor model of the connection between the sensor measurements and the tissue glucose level is provided and a state transition model is provided, which comprises a model for the connection between the tissue glucose level and the blood glucose value and, in a further process step, there is a quantification of the blood glucose level of the organism by means of the sensor model and the state transition model dependent on the sensor measurements, where it is essential to use a Moving Horizon Estimation Method.

The Moving Horizon Estimation method, MHE for short, is known in principle for the evaluation of measurement signals that are present as series of statistical values. Research by the applicant has shown that, in contrast to the processes used heretofore, the use of a Moving Horizon Estimation method offers significant advantages for the evaluation of the sensor measurements for the quantification of blood glucose levels, both in terms of the accuracy of the estimation and also in terms of the flexibility relating to the assumptions for the models and also in terms of the speed in providing the blood glucose levels. Preferably the Moving Horizon Estimation method results in a current blood glucose concentration and a retrospective blood glucose concentration, in which the retrospective blood glucose concentration is preferably determined taking into account at least one past blood glucose concentration (a previously determined blood glucose concentration over a course of measurements (course of concentrations)). The retrospective blood glucose concentration thus makes possible, in particular, a better reconstruction of the current blood glucose measurement signal as solely the current blood glucose concentration.

The evaluating device here can be a computer, an integrated circuit or something similar, which is particularly designed for optimized calculation of the trace of a matrix, for example. The device and/or evaluating device can be designed as a portable device with independent sources of energy, batteries for example, or something similar, which allows for efficient operation, and thereby also keeps the energy consumption to carry out the process as low as possible, according to one embodiment of the present invention, in order to make it possible to operate the batteries as long as possible, which enhances the user experience. For this, energy-saving processors, wiring, circuits, interfaces can especially be used, particularly wireless interfaces or the like. The execution of the process here, with respect to its parameters, can be adapted, for example, to the underlying device, for example, the evaluating device, for example, with respect to the evaluation horizon and/or the noise horizon, which will be described subsequently, in order to achieve, on the one hand, sufficient accuracy and, on the other hand, a long running time.

One of the advantages obtained is that an estimation of the current glucose level in the transport fluid, particularly blood, is provided with efficiency in terms of time and computational resources. In addition, there is an advantage that the flexibility as compared to known processes is significantly increased, because limitations to certain sensor models and/or state transition models are eliminated. A further advantage is that not only is the accuracy of the current glucose level increased, but, at the same time, previous glucose levels are likewise improved.

Further features, advantages and embodiments of the invention are described below or are disclosed therein.

According to a preferred embodiment, a Moving Horizon Estimation method is carried out to provide the current glucose level in step d) and applied to previously provided glucose measurements and at least one previous tissue glucose measurement. In particular, this makes possible an efficient determination of the current glucose levels on the basis of previously measured glucose levels.

According to another preferred embodiment, the sensor model is provided in form of a linear function between measurements and tissue glucose levels. This allows for a particularly efficient and fast calculation of the interstitial tissue glucose levels on the basis of the measurements of the series of measurements with sufficient accuracy at the same time.

According to another preferred embodiment, the sensor model is provided in form of a non-linear function between measurements and tissue glucose levels. Here, for example, the following sensor models can be provided, in which y represents the measurement, IG represents the glucose concentration and a, b, c or A, b, c represent sensor parameters:

y=c−a*b/(IG+b)

Alternatively, the following non-linear sensor model is possible:

y=(A*b+c*IG)/(IG+b)

The advantage of this is higher accuracy of the calculated interstitial tissue glucose levels based on measurements of the series of measurements, particularly with sensor devices with affinity binding sensors or optical sensors.

According to a further preferred embodiment, the value for the horizon of the Moving Horizon Estimation method for providing the current glucose level is selected as less than or equal to 10. This enables a particularly efficient and fast calculation of the interstitial tissue glucose levels on the basis of the measurements of the series of measurements with sufficient accuracy at the same time.

According to a further preferred embodiment, a variance of the measurement noise and/or process noise is estimated, particularly at least on a regular basis. This makes it possible to provide noise measurements simply and quickly and thereby, over all, an accurate determination of the current glucose level.

According to a further preferred embodiment, the variance of the measurement noise and/or the variance of the process noise is estimated or especially interpolated and/or weighted, preferably using an exponential smoothing. In this case, any noise levels that vary depending on time can be adapted or updated, which further improves the overall accuracy in determining the current glucose levels.

According to a further preferred embodiment, the measurements that have only partially used to calculate the estimation of the measurement noise and/or the process noise can be temporarily stored, and measurements that have not been temporarily stored and that are needed can be interpolated using the measurements that have been stored. Therefore, for example, it is possible, for the determination of measurement noise levels and/or process noise levels to temporarily store necessary and calculation-intensive measurements at least partially and to make them available for subsequent measurements, which overall decreases the amount of calculation needed to determine the current glucose level without significantly lessening the accuracy thereof.

According to a further preferred embodiment, a selection is made of a quantity of the previous measurements, which is greater than the measurement for the horizon of the Moving Horizon Estimation methods, in particular at least twice as great, preferably at least by the factor 5. Thereby the accuracy of the estimation of process and/or measurement noise in relation to the current glucose level is determined and improves the overall accuracy of the determination or quantification of the current glucose level.

According to a further preferred embodiment, the variance of the measurement noise and/or the variance of the process noise is regularly adjusted based on the level of the sum of the horizon of the Moving Horizon Estimation method and the number of the previous measurements to calculate the estimation of the measurement noise and/or the process noise. This ensures that an efficient adjustment of the noise measurements at any given time will take place at regular intervals, on the one hand, in order to achieve sufficient accuracy of the current glucose level and, on the other hand, to prevent unnecessary adjustments or updates, which do not result in an increase in the accuracy of the current glucose level or do so insignificantly.

According to a further preferred embodiment, measurements provided are filtered by means of a filter function, whereby, by means of the filter function, errors, especially measurement errors, are suppressed by the sensor device. By means of the filter function, erroneous measurements can be simply sorted out, for example sensor errors or outliers in the measurements; this means that they are not taken into account in the further calculation of the current glucose level.

According to a further preferred embodiment, measurement noise measurements are weighted by means of the filter function. Thereby underestimation and overestimation of measurement noise levels is prevented: underestimation results in extremely erroneous signals or measurements, while overestimation results to an overly smooth course of measurements of the series of measurements. Overall, the accuracy is thereby further improved.

According to a further preferred embodiment, in order to determine errors of the sensor device, the gradient of the increase in a current tissue glucose level and/or the current tissue glucose level is evaluated. Alternatively, or additionally, this can also be carried out with a current glucose level and/or its gradient of the increase in the current glucose level of the transport fluid. This provides a simple and at the same time reliable and efficient recognition of errors of the sensor device.

According to a further preferred embodiment, measurements that are provided below a low threshold level that can be preset and/or above a high threshold level that can be preset are discarded by means of the filter function, particularly the low and high threshold level corresponds to physiological limits, preferably where the low threshold level presents a value between 10-50 mg/dL, particularly 30 mg/dL and the high threshold level presents a value between 100-600 mg/dL, preferably 450 mg/dL. By means of appropriate threshold levels, erroneous sensor measurements, which include both glucose measurements in the transport fluid, especially in the blood, and also tissue glucose measurements, are cut out in an advantageous manner through a weighting matrix, most advantageously a diagonal weighting matrix, for further calculation. Advantageously the weighting matrix functions in such a way that the erroneous measurements of the sensor device can be weighted with the factor 0, while all other measurements are weighted with the factor 1. The erroneous measurements, for example outliers in the sensor measurements, are determined by way of the absolute glucose concentration in the transport fluid, especially blood, as well as its rate of change or its gradients. In the first case cited, preferably the physiological bounds of a blood glucose concentration are introduced, wherein a blood glucose concentration ranging from 10 mg/dL to 600 mg/dL, preferably 20 mg/dL to 500 mg/dL, most preferably 30 mg/dL to 450 mg/dL, is assumed. Measurements outside these physiological bounds are weighted as erroneous sensor measurements with the factor 0. The gradient of the glucose concentration in the transport fluid, particularly in the blood, or the rate of change of the glucose concentration in the transport fluid, particularly in the blood, can likewise be determined and its level can be compared with a physiologically realistic rate of change. The quantitative rate of change of the glucose concentration in the transport fluid, particularly in the blood, is accordingly a value from 0.1 mg/dL per min to 15 mg/dL per min, preferably a value from 0.5 mg/dL per min to 10 mg/dL per min, most preferably a value from 1 mg/dL per min to 3 mg/dL per min.

According to a further preferred embodiment, a calibration of the measurements of the sensor device is executed after execution of step d). Thereby the use of a non-calibrated tissue glucose measurement is possible, which has the advantage that the calibration does not necessarily have to take place before the Moving Horizon Estimation method is carried out, but rather it can likewise take place after the Moving Horizon Estimation method. A further advantage of the use of non-calibrated tissue glucose measurements is that the non-calibrated tissue glucose measurements present a higher correlation to self-monitoring blood glucose concentration and thereby, for example, the parameters of the sensor model can be advantageously determined more simply and precisely.

According to a further preferred embodiment, the state transition model includes a diffusion model for time-dependent modeling of the diffusion process of glucose from the transport fluid into the surrounding tissue. By means of a diffusion model, especially based on a diffusion constant, a simple and at the same time less computationally intensive modeling of the attenuation and the time delay between the glucose level in the transport fluid, especially in the blood, and the tissue glucose level is provided.

According to a further preferred embodiment, sensor model parameters of the sensor model and/or state transition parameters of the state transition model are estimated and/or updated, at least on a regular basis. The advantage of this is that, overall, the accuracy is thereby increased for the determination of the current glucose level; likewise, parameters of the model in question can be flexibly adjusted to changing circumstances or influences.

Other important features and advantages of the invention result from the dependent claims, from the drawings and the corresponding description of the figures using the drawings.

It is understood that the above-mentioned features and features yet to be explained, not only may not only be used in the respectively indicated combination, but rather also in other combinations or alone, without departing from the scope of the present invention.

Preferred designs and embodiments of the invention are presented in the drawings and are explained further in the description below. All remodeling steps of equations, assumptions, processes for solution etc. can be used separately without going beyond the scope of the invention.

In a diagrammatic form

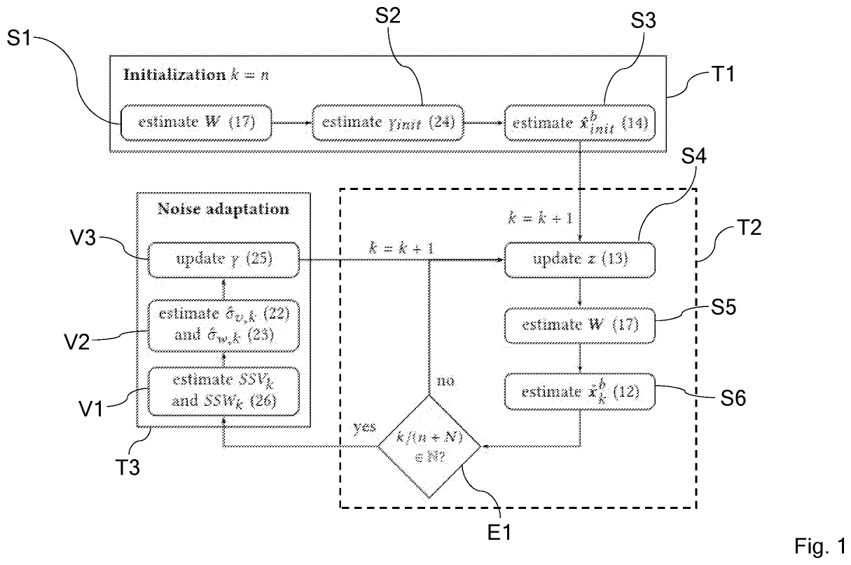

FIG. 1 shows steps of a process according to an embodiment of the present invention;

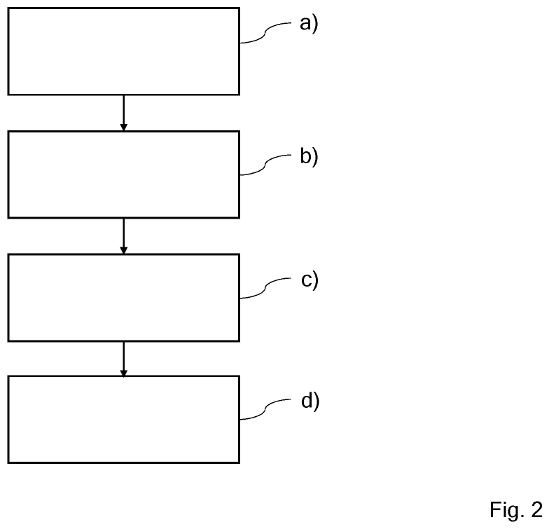

FIG. 2 shows steps of a process according to an embodiment of the present invention; and

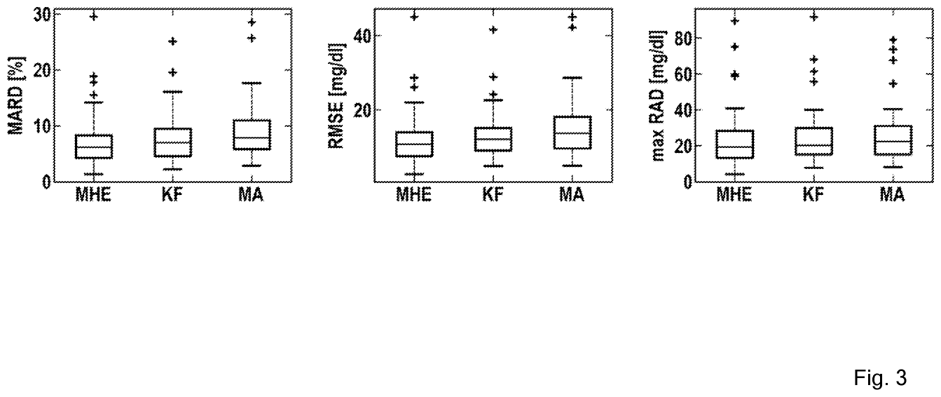

FIG. 3 shows a comparison of a process according to an embodiment of the present invention with already known processes.

FIG. 1 shows in detail steps for determining the glucose concentration in the blood based on the Moving Horizon Estimation method, where the variation of the process noise and the measurement noise is adjusted.

In an initial phase T1, there is the initializing of the process by means of the steps S1-S3 explained below. After the initializing, in a second phase T2, in discreet steps in time, based on the Moving Horizon Estimation method, the determination of the glucose concentration in the blood by means of steps S4-S6 is explained below, as well as a decision step E1. Parallel to this, in third phase T3, an adjustment of measurement and process noise with steps V1-V3 explained below is performed.

Before going into individual phases T1-T3 and their steps in detail, first the principles for carrying out the Moving Horizon Estimation method are explained below. The Moving Horizon Estimation method that is used below is a method for estimating a state by minimizing a so-called cost function, which is carried out on a moving time window of n discreet steps in time. Here a discreet time system is defined.

x_{K}=f(xₖ₋₁,wₖ₋₁)

y_{K}=h(xₖ,vₖ)

in which x_{K} is the vector of the state variable and y_{K} is the measurement vector. Furthermore, the cost function includes the weighted norm of the measurement noise v_{K} and process noise wₖ of horizon n at time k.

The optimizing problem thereby takes the following form:

$${min}_{{\{ x \}}_{j \vert k}^{k \vert k}} {\sum }_{j = k - N + 1}^{k} \frac{1}{{σ}_{υ , k}} {‖ {v}_{j \vert k} ‖}^{2} + \frac{1}{{σ}_{w , k}} {‖ {w}_{j - 1 \vert k} ‖}^{2} \qquad ( 2 )$$

in which {x}_{j|k}^{k|k} are the estimated states x_{k-N+1}, . . . , xₖ for time k and σ_{w,k}²=var(wₖ) and σ_{v,k}²=var(vₖ) are the variances of the process noise or measurement noise. Process and measurement noises are uncorrelated with mean value 0, but are not necessarily Gaussian distributed.

For the modeling of the diffusion process between blood and the surrounding tissue, in particular, the following connection is assumed:

$$\frac{d i ( t )}{d t} = \frac{1}{τ} ( b ( τ ) - i ( t ) ) \qquad ( 3 )$$

in which i(t) represents the tissue glucose signal, b(t) represents the blood glucose signal and τ the time constant of the diffusion process. The sensor signal, in particular, is taken as a linear model with measurement y(t):

y(t)=p₀i(t)+p₁

However, it is also possible to use a non-linear model, for example of the form f(iₖ)=(p₁*iₖ)/(p₀+iₖ).

Under the assumption that output signal y(t) is linear in it(t), an ideal and calibrated blood glucose signal x^{b}=p₀ b(t)+p₁ and an ideal and calibrated tissue glucose signal xⁱ=p₀ i(t)+p₁ can be introduced. Under the further assumption that the sensor parameters p₀ and p₁ change only slowly, the diffusion is likewise valid for the non-calibrated signals

$$\frac{d {x}^{i} ( t )}{d t} = {p}_{0} \frac{d i ( t )}{d t} = \frac{1}{τ} ( {x}^{b} ( t ) - {x}^{i} ( t ) ) \qquad ( 4 )$$

If this equation is now modeled in time steps Δt and the blood glucose concentration x^{b} is modeled with an autoregressive model, one arrives at the following discreet state representation:

$${x}_{k + 1}^{b} = 2 {x}_{k}^{b} - {x}_{k - {1}^{+}}^{b} {w}_{k} \\ {x}_{k + 1}^{i} = {x}_{k}^{i} + \frac{1}{τ} ( {x}_{k}^{b} - {x}_{k}^{i} ) \\ {y}_{k} = {x}_{k}^{i} + {υ}_{k} \qquad ( 5 )$$

For the above-name alternative non-linear model, the following state space representation would be valid:

$${b}_{k + 1} = 2 {b}_{k} - {b}_{k - 1} + {w}_{k} \\ {i}_{k + 1} = {i}_{k} + \frac{1}{τ} ( {b}_{k} - {i}_{k} ) \\ f ( {i}_{k} ) = \frac{{p}_{1} \cdot {i}_{k}}{{i}_{k} + {p}_{0}} \\ {y}_{k} = f ( {i}_{k} ) + {v}_{k} \\ {w}_{k} = {b}_{k + 1} - 2 {b}_{k - 1} \\ {v}_{k} = {y}_{k} - \frac{{p}_{1} {i}_{k}}{( {p}_{0} + {i}_{k} )}$$

Below the linear sensor model described above will again be assumed. Therefore, as soon as the ideal non-calibrated blood glucose level xₖ^{b} is estimated, the blood glucose concentration can be determined for the linear model by means of:

$${b}_{k} = \frac{{y}_{k}^{b} - {p}_{1}}{{p}_{0}} \qquad ( 6 )$$

The Moving Horizon Problem formulation in equation (2) is now solved through optimizing the formulated noise-free blood glucose signal

x_{k-N+1|k}^{b}, . . . ,x_{k|k}^{b}

Below a matrix notation will be used with N dimensional vectors, which include the past state variables or measurements of the sensor for point in time k.

j=k−N−1, . . . ,k

The tissue glucose signal xₖⁱ is thus described as

xₖⁱ=Axₖ^{b}+Bzₖ (7)

in which zₖ:=(x_{k-N}ⁱ, x_{k-N-1}^{b}, x_{k-N}^{b})^{T} the initial states and matrices A and B are defined as follows

$$A = \frac{1}{τ} ( \begin{matrix}0 & 0 & 0 & … & 0 \\ 1 & 0 & 0 & … & 0 \\ a & 1 & 0 & … & 0 \\ ⋮ & ⋮ & ⋱ & ⋱ & ⋮ \\ {a}^{N - 2} & … & a & 1 & 0\end{matrix} ) , B = ( \begin{matrix}a & 0 & {a}^{0} \\ ⋮ & ⋮ & ⋮ \\ {a}^{N} & 0 & {a}^{N - 1}\end{matrix} )$$

with

$$∝ = 1 - \frac{1}{τ}$$

The measurement noise vₖ=(v_{k-N+1|k}, . . . , v_{k|k})^{T} is given through

vₖ=yₖ−xₖⁱ (8)

and the process noise wₖ=(w_{k-N|k}, . . . , w_{k-1|k})^{T} is defined as

$${w}_{k} = \underbrace{( \begin{matrix}0 & 0 & 0 & … & 0 \\ - 2 & 1 & 0 & … & 0 \\ 1 & - 2 & 1 & … & 0 \\ ⋮ & ⋱ & ⋱ & ⋱ & ⋮ \\ 0 & … & 1 & - 2 & 1\end{matrix} )}_{C ∈ {R}^{N \times N}} {x}_{k}^{b} + \underbrace{( \begin{matrix}0 & 1 & - 2 \\ 0 & 0 & 1 \\ ⋮ & ⋱ & ⋮ \\ 0 & … & 0\end{matrix} )}_{D ∈ {R}^{N \times 3}} {z}_{k} \qquad ( 9 )$$

The formulation of the Moving Horizon Problem for the linear sensor model is then provided through

$${min}_{{x}_{k}^{b}} ( {‖ {w}_{k} ‖}_{{Q}_{k}^{- 1}}^{2} + {‖ {v}_{k} ‖}_{{R}_{k}^{- 1}}^{2} ) \qquad ( 10 )$$

with the weight matrices Qₖ=cov(wₖ) and Rₖ=cov(vₖ), which correspond to the covariance matrices of the process noise and the measurement noise. For the alternative, non-linear sensor model that was described, the following optimization problem is provided:

$${min}_{{b}_{k - N} … {b}_{k}} \{ {\sum }_{j = k - N - 1}^{k - 1} {‖ {w}_{k} ‖}_{\frac{1}{{σ}_{w}^{2}}}^{2} + {\sum }_{j = k - N}^{k} {‖ {v}_{n} ‖}_{\frac{1}{{σ}_{v}^{2}}}^{2} \} ,$$

in which the variants σ_{w}² and σᵥ² must be determined or provided. This optimization problem can generally not be solved directly, but rather by means of iterative solution processes. The determination or updating of the sensor parameters of the nonlinear model then results from the self-monitoring measurements bgi(t=ti):

$${min}_{{p}_{0} , {p}_{1}} {\sum }_{i = 1}^{N} {‖ {bg}_{i} - \hat{b} ( t = {t}_{i} , {p}_{0} , {p}_{1} ) ‖}^{2}$$

Below the linear sensor model will now again be assumed. After insertion of equations (8) and (9) into the Moving Horizon Problem for the linear model, the following quadratic problem results:

$${min}_{{x}_{k}^{b}} \{ {x}_{k}^{{b}^{T}} ( {A}^{T} {R}^{- 1} A + {C}^{T} {Q}^{- 1} C ) {x}_{k}^{b} - ( {( {y}_{k} - B {z}_{k} )}^{T} {R}^{- 1} A - {z}_{k}^{T} {D}^{T} {Q}^{- 1} C ) {x}_{k}^{b} \} \qquad ( 11 )$$

which can be solved through matrix inversion

\hat{x}ₓ^{b}=(A^{T}R⁻¹A+C^{T}Q⁻¹C)⁻¹·(A^{T}R⁻¹(yₖ−Bzₖ)−C^{T}Q⁻¹Dzₖ)=Hyₖ+Gzₖ (12)

or by taking into account known processes for the solution of quadratic optimization problems.

The initial states zₖ are determined according to the solution of the previous estimation steps:

$${z}_{k} = ( \begin{matrix}{x}_{k - N}^{i} \\ {x}_{k - N - 1}^{b} \\ {x}_{k - N}^{b}\end{matrix} ) = ( \begin{matrix}{\hat{x}}_{k - N \vert k - 1}^{i} \\ {\hat{x}}_{k - N - 1 \vert k - 2}^{b} \\ {\hat{x}}_{k - N \vert k - 1}^{b}\end{matrix} ) . \qquad ( 13 )$$

In the event of initialization with k+N, the initial states must be estimated. Accordingly, the initial points are added to the optimization vector xᵢₙᵢₜ=(zₖ, xₖ^{b}). The optimization problem can accordingly then be transcribed with Aᵢₙᵢₜ=[BA] and Cᵢₙᵢₜ=[DC] and by replacing Bᵢₙᵢₜ and Dᵢₙᵢₜ with zero matrices.

The solution of the altered optimization problem is then

$${\underbrace{{\hat{x}}_{i n i t} = {( {A}_{init}^{T} {R}_{init}^{- 1} {A}_{init} + {C}_{i n i t}^{T} {Q}_{i n i t}^{- 1} {C}_{i n i t} )}^{- 1} {A}_{i n i t}^{T} {y}_{k}}}_{{H}_{init}} \qquad ( 14 )$$

In contrast to other estimation processes, by means of the Moving Horizon Estimation method, both a current value \hat{x}_{k|k}^{b} and previous values \hat{x}_{j|k}^{b} (j>k-N+1) are estimated. The updating of the blood glucose level or signal, not only with the current estimation value, but rather likewise with the estimation values in the overall (past) horizon/time window \hat{x}_{j|k}^{b} (j>k-N+1) results in:

\hat{X}^{b}(k−N+1, . . . ,k)=\hat{x}ₖ^{b}

\hat{X}ⁱ(k−N+1, . . . ,k)=\hat{x}ₖⁱ=A\hat{x}ₖ^{b}+Bzₖ. (15)

Here \hat{X}^{b}(i) for i≤k-N includes only the estimation values

\hat{x}_{k-N+1|k}^{b}

Since CGM systems are typically sensitive to mechanical disturbances that can result in erroneous measurements, such erroneous sensor measurements can be taken into account through weighting the measurement noise with a weighting matrix W.

In equations (11) and (12) described above, a weighted, inverse covariance matrix can be introduced,

$$\underbrace{{R}^{- 1} = \frac{1}{N} {\sum }_{i = k - N}^{k} {w}_{i} ( \begin{matrix}{w}_{k} & & 0 \\ & ⋱ & \\ 0 & & {w}_{k - N}\end{matrix} ) {R}^{- 1 }}_{W} \qquad ( 16 )$$

in order to be able to estimate the solution of the optimization problem. Here the diagonal entries of the weighting matrix W corresponding to erroneous measurements are set to 0.

$${w}_{i} = \{ \begin{matrix}0 & sensor error or outliers \\ 1 & else\end{matrix} \qquad ( 17 )$$

In order to detect sensor errors or measurement outliers, particularly the gradient in the corresponding tissue glucose level as well as the current tissue glucose value can be used. Alternatively, or additionally, it is possible to apply high or low threshold values for the measurement signals or the measurement values of the sensor, and to classify measurement values that lie outside low and high threshold values as erroneous.

To efficiently carry out the Moving Horizon Estimation method, it is especially necessary to be familiar with the variance of the measurement noise σ_{v,k}² and the process noise σ_{w,k}². Generally, these two parameters are unknow and must be estimated. Moreover, these parameters change over the course of time. An adjustment or updating of the variances therefore results directly in a change, particularly an improvement in the quality of the estimation through the Moving Horizon Estimation method. If, however, the measurement noise is estimated too low, this results in a very noisy measurement signal and thereby to erroneous measurements. If, on the other hand, the measurement noise is estimated too high or the process noise is estimated too low, this results in a time-delayed estimation, which likewise reduces the accuracy of the determination of the current glucose level.

Below a process will now be described to predict the measurement noise variance of the measurement noise and the process noise variance of the process noise. Furthermore, a process will be described below for easily adjusting or updating the variances in any given case.

The principle for this process is that every degree of freedom is equivalent to every other degree of freedom, as described, for example, in the non-patent literature by Grace Wahba “Bayesian ‘Confidence Intervals’ for the Cross-Validated Smoothing Spline” (Journal of the Royal Statistical Society: Series B (Methodological) 45, (1983), 133-150).

Under the assumption that the process noise w_{j-1|k} and the measurement noise v_{j|k} of a horizon are of length n and j=k−n+1, . . . k is part of a distribution with variance σ_{w,k} or σ_{v,k}, the covariance matrices Rₖ and Qₖ correspond to

Rₖ=σ_{v,k}²I and Qₖ=σ_{w,k}².

equation (12) is thereby simplified to

H(γₖ)=(A^{T}A+γₖC^{T}C)⁻¹A^{T}

and

G(γₖ)=(A^{T}A+γₖC^{T}C)⁻¹(B−C^{T}D)

in which

$${γ}_{k} = \frac{{σ}_{v , k}^{2}}{{σ}_{w , k}^{2}}$$

is the quotient of the variance of the process noise and of the measurement noise.

In the next step, equations (7) and (12) are inserted into the definition of measurement noise (8) and of process noise (9):

w=CH(γₖ)γₖ+(CG+D)zₖ

v=(I−AH(γₖ)γₖ−(AG+B)zₖ

Under the assumption that the variance of initial points zₖ is equal to zero, the covariance matrices have the following form

cov(wₖ)=CH(γₖ)cov(γₖ)H^{T}(γₖ)C^{T}

cov(vₖ)=(I−AH(γₖ))cov(γₖ)(I−AH(γₖ))^{T} (18)

Furthermore, the covariance of the non-calibrated noise-free blood glucose signal cov(xₖ^{b})=σ_{w}²C⁻¹(C⁻¹)^{T} and the covariance of the ideal, non-calibrated tissue glucose signal cov(xₖ^{b})=σ_{w}²AC⁻¹(C⁻¹)^{T}A^{T} is dependent only on the variance matrix of the process noise. Since the covariance matrix of the measurement signal comprises the covariance matrix of the measurement noise and the non-calibrated, noise-free tissue glucose signal, it thus follows

cov(yₖ)=σᵥ²I+σ_{w}²A(C⁻¹)^{T}C⁻¹A (19)

Inserting equation (19) now into equation (18) and performing a matrix inversion, there results the covariance matrix of the process noise and of the measurement noise in the following manner:

cov(vₖ)=σᵥ²(I−AH(γₖ)) (20)

cov(wₖ)=σ_{w}²CH(γₖ)AC⁻¹ (21)

The expected value of the sum of the squared measurement error SSVₖ=vₖ^{T}vₖ can be transcribed to

E(SSVₖ)=E(vₖ^{T}vₖ)=E(tr(vₖvₖ^{T})=E(tr(cov(vₖ)))

The variance of the measurement noise then results in the following manner:

$${σ}_{v , k}^{2} = \frac{E ( S S {V}_{k} )}{N - s ( {γ}_{k} )}$$

in which s(γₖ)=tr(AH(γₖ)) is

Thereby a consistent estimated value for the variance of the measurement noise \hat{σ}_{v,k}² is provided.

$${\hat{σ}}_{υ , k}^{2} = \frac{SS {V}_{k}}{N - s ( {γ}_{k} )} \qquad ( 22 )$$

The variance of the process noise can be determined in a similar manner as the variance of the measurement noise.

$${\hat{σ}}_{w , k}^{2} = \frac{{SSW}_{k}}{s ( {γ}_{k} )} \qquad ( 23 )$$

In this respect, the expected values of the sum of the quadratic process errors SSWₖ=wₖ^{T}wₖ and equation (21) are used. Since the value γₖ must fulfill the equation,

$${{\hat{γ}}_{k}}^{=} \frac{{\hat{σ}}_{υ , k}^{2}}{{\hat{σ}}_{w , k}^{2}} = \frac{s ( {\hat{γ}}_{k} ) SS {V}_{k} ( {\hat{γ}}_{k} )}{( n - s ( {γ}_{k} ) ) SS {W}_{k} ( {\hat{γ}}_{k} )}$$

the optimal \hat{γ}ᵢₙᵢₜ is estimated with a derivation-free optimization process, such as, for example, described in the non-patent literature Rios and Sahinidis “Derivative-free optimization: a review of algorithms and comparison of software implementations” (Journal of Global Optimization 56, 3 (2013), 1247-1293):

$${min}_{γ} {‖ γ - \frac{s ( γ ) S S V ( γ , {x}_{i n i t}^{b} )}{( n - s ( γ ) ) S S W ( γ , {x}_{i n i t}^{b} )} ‖}^{2} \qquad ( 24 )$$

After that,

$${\hat{γ}}_{k} = \frac{{\tilde{σ}}_{υ , k}^{2}}{{\tilde{σ}}_{w , k}^{2}}$$

is adjusted and the process noise and the measurement noise after n+N time is updated

\tilde{σ}_{v,k}²(1−η)\hat{σ}_{v,k}²+η\tilde{σ}_{v,k-n}²

\tilde{σ}_{w,k}²(1−η)\hat{σ}_{w,k}²+η\tilde{σ}_{w,k-n}² (25)

taking into account a smoothing factor η.

The length of the noise adjustment horizon n does not have to agree with estimation horizon N. A longer estimation horizon N significantly increases the computational effort; however, it shows only a slightly improved estimation accuracy. Since the estimation accuracy of the variances is strongly correlated with the number of data points, especially noise adjustment horizon n will be selected significantly greater than estimation horizon N.

Variances σ_{w,k}² and \hat{σ}_{v,k}² are estimated using equations (22), (23) and the sum of the squared process errors or the corresponding sum of the squared measurement errors:

$$S S {V}_{k} = {{\sum }_{j = k - n + 1}}^{k} {\hat{X}}^{b} ( j ) - 2 {\hat{X}}^{b} ( j - 1 ) + {\hat{X}}^{b} ( j - 2 ) \\ {SSW}_{k} = {{\sum }_{j = k - n + 1}}^{k} {\hat{X}}^{i} ( j ) - y ( j ) . \qquad ( 26 )$$

In the event of erroneous sensor measurements, the noise variances in particular are not updated, because this can result in an erroneous estimation of process and measurement variances σ_{v,k}² and σ_{w,k}².

In order to be able to estimate s(γₖ)=tr(AH(γₖ)), a higher computational effort is necessary. Since matrix A is dependent only on pre-defined matrices A and C and on γ, it is possible to draw up a table for γ and the relevant range of the quotient and to use an interpolation of the values of the table for the current value of γ. Therefore, even on a computer or on a tablet, a satisfactory and sufficiently accurate estimation can be made possible in a short time with little computational effort.

To summarize, especially in initial step S1, matrix W can be estimated during initialization phase T1 according to equation (17). On the basis of matrix W, in a second step S2, the ratio of the variances of process noise and measurement noise is estimated according to equation (24). In a third step S3, the initial value is estimated according to equation (14).

On the basis of the estimated initial value, in a fourth step S4 during initial phase T2, the initial states are first determined according to equation (13) and, using the initial states, matrix W is estimated again in a fifth step S5 analogously to step S1 according to equation (17). In a sixth step S6, the initial value is estimated according to equation (12). Afterwards, in step E1, it is determined whether the quotient from time k and the sum from estimation horizon N and noise-adjustment horizon n yields an integer larger or equal to one or not. If this is not the case, the time index k is increased by one and steps S4 to S6 as well as E1 are then carried out again. If this, on the contrary, is the case, a noise adjustment T3 is carried out with steps V1 to V3. In noise adjustment T3, in an initial step V1, the sum of the squared process errors or measurement errors is determined according to equation (26). Using these, the corresponding variances of measurement noise and process noise is then determined according to equations (22) and (23) in a second step V2 and then the value for is updated according to equation (25). After this, steps S4 to S6 as well as E1 are then carried out again.

On the basis of the estimated initial value, in a fourth step S4 during initial phase T2, the initial states are first determined according to equation (13) and, using the initial states, matrix W is estimated again in a fifth step analogously to step S1 according to equation (17). In a sixth step S6, the initial value is calculated according to equation (12). Afterwards, in step E1, it is determined whether the quotient from time k and the sum from estimation horizon N and noise-adjustment horizon n yields a integer larger or equal to one or not. If this is not the case, the time index k is increased by one and steps S4 to S6 as well as E1 are then carried out again. If this, on the contrary, is the case, a noise adjustment T3 is carried out with steps V1 to V3. Here, in an initial step V1, the sum of the squared process errors or measurement errors is determined according to equation (26). Using these, the corresponding variances of measurement noise and process noise is then determined according to equations (22) and (23) in a second step V2. Then the value for γ is updated according to equation (25). After this, steps S4 to S6 as well as E1 are then carried out again.

In FIG. 2, steps of a process according to one embodiment of the present invention are shown.

In detail, FIG. 2 shows a process to, preferably continuously, determine a current glucose level in a transport fluid, particularly blood, of an organism. The method comprises at least the following steps:

In step a), using a sensor device, a series of measurements is determined, comprising at least two measurements separated in time for a tissue glucose level, in which the tissue surrounding the transport fluid.

In a further step b), the tissue glucose level is determined using the series of measurements provided, based on a sensor model, in which, by means of a sensor model, measurements of the sensor device are correlated to the tissue glucose levels while taking into account measurement noise.

In a further step c), a state transition model is provided, in which, by means of the state transition model, at least one glucose level in the transport fluid is correlated to the tissue glucose levels that have been determined while taking into account process noise.

In a further step d), the current glucose level is determined, based on the provided state transition model and the tissue glucose level that has been determined, in which, at least step d), particularly steps b)-d), is carried out using at least one Moving Horizon Estimation Method.

FIG. 3 shows a comparison of a process according to one embodiment of the present invention with already known processes.

Below a comparison of different blood glucose estimation processes is explained and the result is presented in FIG. 3. In detail, the estimation processes are carried out with the same sensor device—Fiber sense—being known from the non-patent literature KUster, Nikolaus et al. “First Clinical Evaluation of a New Percutaneous Optical Fiber Glukose Sensor for Continuous Glukose Monitoring in Diabetes” (Journal of Diabetes Science and Technology 7, 1 (2014), 13-23), which determines the blood glucose content on the basis of fluorescence measurements exhibiting a sampling rate every 2 minutes. An estimation of the blood glucose concentration by means of a Moving Horizon estimation method according to an embodiment of the present invention is compared here with two other estimation methods, the Kalman-Filtering KF as well as with a smoothed sensor signal with a sliding mean average filter MA. Additionally, the effect of different parameters on the blood glucose estimation is explained. Here the data were gathered using eight type 1 and eight type 2 diabetes patients, in which the corresponding CGM sensor of the sensor device was carried over 28 days. On days 1, 7, 15 and 28, reference data were ascertained every 10 minutes over a time of 4 houses with the use of Yellow Springs Instrument (YSI) 2300 STAT Plus Glukose analyzer (YSI Life Sciences, Yellow Springs, Ohio).

Furthermore, the horizon for the Moving Horizon Estimation was set at N=10 and the horizon for the noise adjustment was set at n−50. The Kalman filtering here is based on equation (5). A diffusion constant or respectively a time constant of τ=6 minutes was assumed for both processes. Overall, the filtered signals of the CGM system, namely \hat{x}ₖ^{b}, were produced by means of the Moving Horizon Estimation method as well as by means of the Kalman filter and compared to the smoothed signal by means of the sliding mean average, based on their agreement at any given time with the measured reference data during the clinical monitoring. For the evaluation of the three different estimation methods, three evaluation parameters are used, first the mean absolute relative difference (MARD), the root of the mean squared error (RMSE) and the maximal relative absolute difference (maxRAD) of the four clinical measurements of all 16 patients.

In the following table, for the evaluation of the media, the 25% quartile—Q1—and the 75% quartile—Q3—of the corresponding three evaluation parameters are shown.

| method | MARD [%] | RMSE [mg/dl] | RMSE [mg/dl] |
|---|---|---|---|
| MHE | 6.1 [4.3, 9.3] | 8.2 [5.7, 11.2] | 19.0 [13.0, 29.5] |
| KF | 7.1 [4.6, 9.6] | 9.6 [6.9, 12.5] | 20.0 [14.9, 31.2] |
| MA | 7.8 [5.3, 11.6] | 11.6 [8.1, 15.2] | 22.8 [14.3, 31.9] |

It is to be understood that the Moving Horizon Estimation method leads to the best results of all three evaluation parameters, followed by the Kalman filter signal KF. The signal MA that is smoothed by means of the sliding mean average, which represents a filtered tissue glucose signal, does not take into account the diffusion process between the blood glucose concentration and the tissue glucose concentration and leads to correspondingly poor results.

To represent the effect of the calibration of the sensor, different calibration methods are explained below and compared with one another.

Here, past estimation results \hat{x}_{k-N+1|k}^{b} can be used. This signal will be referred to below as \hat{X}^{b} signal pMHE, comprising earlier estimation results and providing small parameters compared with the results of the Moving Horizon signal, which comprises only the current estimation values \hat{x}_{k|k}^{b} (median MARD=5,1%, median RMSE=7.1 mg/dL and median maxRAD=14.2 mg/dL).

The previous, estimated values \hat{X}_{k-N+1|k}^{b} accordingly improve the blood glucose measurements and result in an improvement in the sensor calibration. The increased accuracy results from an improved consideration of the time delay. An “over-shoot” because of rapid changes in the blood glucose concentration or because of noise is likewise reduced.

An effect on the accuracy of the blood glucose estimation using the CGM signal on the calibration error will be described below. For this, a so-called two-point calibration method is used. Two reference measurements b₁, b₂ and the corresponding blood glucose results in time (\hat{x}₁^{b}, \hat{x}₂^{b}) are used to calculate the sensor parameters according to

$${p}_{0} = \frac{{b}_{2} - {b}_{1}}{{\hat{x}}_{2}^{b} - {\hat{x}}_{1}^{b}} and {p}_{1} = {b}_{1} - {p}_{0} {\hat{x}}_{1}^{b}$$

Every reference combination and for every estimated, non-calibrated blood glucose signal (MHE, pMHE, KF and MA), the sensor parameters are identified and the blood glucose concentration is calculated. Table 2 that follows

| method | MARD [%] | RMSE [mg/dl] |
|---|---|---|
| pMHE | 10.1 [5.5, 21.5] | 18.2 [10.5, 37.2] |
| MHE | 12.0 [6.8, 25.6] | 21.5 [12.7, 43.2] |
| KF | 12.6 [7.4, 26.7] | 23.0 [14.1, 45.0] |
| MA | 14.3 [8.3, 29.3] | 26.0 [16.5, 50.0] |

shows medians and quartiles of the MARD and RMSe methods for all possible calibrations. From Table 2, it can be seen that the pMHE method exhibits the smallest median and the smallest interquartile distance from MARD and RMSE.

In summary, at least one of the embodiments of the invention has at least one of the following advantages and/or features:
Compensation of the time delay through modeling of the diffusion process and estimation of the blood sugar on a moving horizon in the past (Moving Horizon Estimation method)
Limitation to the physiological range guarantees robustness with respect to outliers.
Adaptive determination of the regulation factors of the problem combines adaptive estimation of the measurement noise and of the state noise.
Adaptation of slowly changing model parameters are additionally possible.
Efficient implementation guarantees a high degree of accuracy with limit computational effort.
Efficient computational estimation over time of the blood sugar on a past horizon.
Adaptation of model parameters
Increase in the robustness of the estimation by the introduction of limitations.
Flexibility with respect to the sensor model, for example, even non-linear sensor models can be used.
Less computational effort to save the limited lifetime of the batteries.
Limitation to the physiological range, guarantees a physiologically reasonable solution.
Optimizing the past horizon improves the estimation of the blood sugar for calibration.

Although the present invention was described using preferred embodiment, it is not limited to these, but rather may be modified in various ways.

1. A process to, preferably continuously, determine a current glucose level in a transport fluid, particularly blood, of an organism, comprising the steps
a) determining, using a sensor device, a series of measurements, comprising at least two measurements separated in time for a tissue glucose level, in the tissue surround the transport fluid,
b) determining the tissue glucose level using the series of measurements given, based on a sensor model, in which, by means of a sensor model, measurements of the sensor device are correlated to the tissue glucose levels while taking into account measurement noise,
c) providing a state transition model, in which, by means of the state transition model, at least one glucose level in the transport fluid is correlated to the tissue glucose levels that have been determined while taking into account process noise, and
d) determining the current glucose level based on the state transition model that has been provided and the tissue glucose level that has been determined,
in which, at least step d), particularly steps b)-d), is carried out using at least one Moving Horizon Estimation Method, preferably a Moving Horizon Estimation method is carried out to provide the current glucose level in step d) and applied to previously provided glucose measurements and at least one previous tissue glucose measurement.

2. The process according to claim 1, characterized in the sensor model is provided in the form of a linear or non-linear function between measurements and tissue glucose levels.

3. The process according to claim 1, characterized in that the value for the horizon of the Moving Horizon Estimation method for providing the current glucose level is selected as less than or equal to 10.

4. The process according to claim 1, characterized in that a variance of the measurement noise and/or the variance of the process noise, particularly at least on a regular basis, is estimated or especially interpolated and/or weighted, preferably where the variance of the measurement noise and/or the variance of the process noise is estimated on the basis of at least one previous value, especially interpolated and/or weighted, using an exponential smoothing.

5. The process according to claim 4, characterized in that measurements that have only partially used to calculate the estimation of the measurement noise and/or of the process noise can be temporarily stored, and measurements that have not been temporarily stored and that are needed can be interpolated using the measurements that have been stored.

6. The process according to claim 3, characterized in that a selection is made of a quantity of the previous measurements, which is greater than the measurement for the horizon of the Moving Horizon Estimation method, in particular at least twice as great, preferably at least by the factor 5.

7. The process according to claim 4, characterized in that the variance of the measurement noise and/or the variance of the process noise is regularly adjusted based on the level of the sum of the horizon of the Moving Horizon Evaluation method and the number of the previous measurements to calculate the estimation of the measurement noise and/or the process noise.

8. The process according to claim 1, characterized in that measurements provided by means of a filter function are filtered by means of a filter function, whereby, by means of the filter function, errors, especially measurement errors, are suppressed by the sensor device, preferably whereby measurements are weighted by means of the filter function.

9. The process according to claim 8, characterized in that, in order to determine errors of the sensor device, the gradient of the increase in a current tissue glucose level and/or the current tissue glucose level is evaluated.

10. The process according to claim 8, characterized in that measurements that are provided below a low threshold level that can be preset and/or above a high threshold level that can be preset are discarded by means of the filter function, particularly the low and high threshold level corresponds to physiological limits, preferably where the low threshold level presents a value between 10-50 mg/dL, particularly 30 mg/dL and the high threshold level presents a value between 100-600 mg/dL, preferably 450 mg/dL.

11. The process according to claim 1, characterized in that a calibration of the measurements of the sensor device is executed after execution of step d).

12. The process according to claim 1, characterized in that the state transition model includes a diffusion model for time-dependent modeling of the diffusion process of glucose from the transport fluid into the surrounding tissue, and/or
sensor model parameters of the sensor model and/or state transition parameters of the state transition model are estimated and/or updated, at least on a regular basis.

13. A device to, preferably continuously, determine a current glucose level in a transport fluid, particularly blood, of an organism, preferably suitable for carrying out a process according to claim 1, comprising
a sensor device, particularly for measuring fluorescence in a tissue surrounding the transport fluid, by means of a fiber optic probe, designed to determine a series of measurements, comprising at least two measurements separated in time for a tissue surrounding the transport fluid,
a provision device designed to provide a state transition model, in which, by means of the state transition model, at least one glucose level in the transport fluid is correlated to the determined tissue glucose levels while taking into account process noise, and to provide a sensor model, in which, by means of a sensor model, measurements of the sensor device are correlated to tissue glucose levels taking into account measurement noise,
an evaluating device designed to determine the tissue glucose level, using the series of measurements provided, based on the sensor model and to determine the current glucose level based on the state transition model that has been provided and the tissue glucose level that has been ascertained, using a Moving Horizon Estimation Method.

14. An evaluation device to, preferably continuously, determine a current glucose level in a transport fluid, particularly blood, of an organism, preferably suitable for carrying out a process according to claim 1, comprising at least one interface to connect a sensor device to provide a series of measurements, comprising at least two measurements separated in time for a tissue glucose level, in which the tissue surrounding the transport fluid,
at least one memory to store a state transition model, in which, at least one glucose level in the transport fluid is correlated to the tissue glucose levels by means of the state transition model while taking into account at least one process noise level, and to store a sensor model, in which, by means of the sensor model, measurements of the sensor device are correlated to tissue glucose levels while taking into account at least one measurement noise, and
a calculating device designed to determine the tissue glucose level, using the series of measurements provided, based on the sensor model and to determine the current glucose level based on the state transition model that has been stored and the tissue glucose level that has been determined, using at least one Moving Horizon Estimation Method.

15. A non-tangible, machine-readable medium for the storage of instructions, which, when carried out on a computer, cause a process, particularly, to continuously determine a current glucose level in a transport fluid, particularly blood, of an organism, to be carried out preferably suitable for carrying out a process according to claim 1, comprising the steps
a) determining, using a sensor device, a series of measurements, comprising at least two measurements separated in time for a tissue glucose level, in the tissue surround the transport fluid,
b) determining the tissue glucose level using the series of measurements given, based on a sensor model, in which, by means of a sensor model, measurements of the sensor device are correlated to the tissue glucose levels while taking into account measurement noise,
c) providing a state transition model, in which, by means of the state transition model, at least one glucose level in the transport fluid is correlated to the tissue glucose levels that have been determined while taking into account process noise, and
d) determining the current glucose level based on the state transition model that has been provided and the tissue glucose level that has been determined,
in which, at least step d), particularly steps b)-d), is carried out using at least one Moving Horizon Estimation Method.
